(19) United States
(12) Patent Application Publication
(10) Pub. No.: US 2012/0078321 A1
(43) Pub. Date: Mar. 29, 2012

(54) SYSTEM AND METHODS OF STIMULATING A HEART

(76) Inventors: Andreas Blomqvist, Taby (SE); Torbjörn Persson, Malmo (SE); Rolf Hill, Jarfalla (SE)

(21) Appl. No.: 13/235,771

(22) Filed: Sep. 19, 2011

Related U.S. Application Data
(60) Provisional application No. 61/427,409, filed on Dec. 27, 2010.

(30) Foreign Application Priority Data
Sep. 23, 2010 (EP) 10178993.1

(51) Int. Cl. A61N 1/365

(52) U.S. Cl. 607/19; 607/17; 607/23

(57) ABSTRACT

A cardiac stimulator is connected to a hemodynamic sensor to sense hemodynamic signals. An optimization module determines recommended AV and VV delays based on IEGM signals. A data processing module determines a delay control parameter for each preset AV delay and VV delay based on the collected hemodynamic signals for respective preset AV delay and VV delay, determines the AV delay setting that corresponds to the maximum delay control parameter and the VV delay setting that corresponds to the maximum delay control parameter, determines an AV delay error correction value as a difference between the AV delay corresponding to the maximum delay control parameter and a recommended AV delay, and determines a VV delay error correction value as a difference between the VV delay corresponding to the maximum delay control parameter and a recommended VV delay.

### RELATED APPLICATION

The present application claims the benefit of the filing date of provisional application Control No. 61/427,409, filed Dec. 27, 2010, the contents of which are incorporated herein by reference.

### BACKGROUND OF THE INVENTION

1. Field of the Invention

The present invention relates generally to implantable medical devices and more particularly to systems and methods for stimulating a heart of a patient.

2. Description of the Prior Art and Related Subject Matter

Heart failure is usually a chronic, long term condition, but may occur suddenly. It may affect the left heart, the right heart, or both sides of the heart. Heart failure may be considered as a cumulative consequence of all injuries and/or stress to the heart over a person's life and the prevalence of heart failure increases constantly. For example, it is estimated that nearly 5 million people in the USA suffer from heart failure and about 400.000 new cases are diagnosed every year. The prevalence of heart failure approximately doubles with each decade of life. One of the most important means of treating heart failure is cardiac resynchronization therapy, CRT. Although CRT is a very effective way of treating heart failure in most patients there is a large percentage for which the CRT has no apparent effect at all. Different estimates of the size of the so called group “non-responders” exist, but it is generally believed to be in the vicinity of 25% of all patients equipped with a CRT device. However, there are numbers reported to be as high as 33% (depending mostly on the definition of CRT response which may vary greatly).

A common method for adapting the therapy (the timing cycles) for non-responders is so called echo-based optimization, which may include M-mode, 2D, 3D and TDI. Echo-based optimization of the timing cycles is often time-consuming and may range from 30 minutes to two hours depending on the scope of the evaluation. Furthermore, echo-based optimization is heavily dependent of the operator, who interprets the displayed echo signals, for accuracy and consistency. Accordingly, there is a need for more reliable, fast, and accurate methods for CRT timing optimization and for patient customized CRT timing optimization.

Device based CRT optimization is likely to be one of the most potent tools in improving CRT efficiency and more specifically fighting non-responders. St. Jude Medical's QuickOpt™ Timing Cycle Optimization is an algorithm that provides IEGM (Intracardiac Electrogram) based AV (Atrial-Ventricular) timing optimization in CRT and ICD (Implantable Cardioverter-Defibrillator) systems and VV (Ventricular-Ventricular) timing optimization in CRT devices in a simple and swift way. QuickOpt™ Timing Cycle Optimization is based on the hypothesis that the point of time for the closure of the Mitral valve can be estimated by measuring the interatrial conduction time (P-wave duration), that the onset of isovolumetric contraction can be measured using the peak of the R-wave and that interventricular conduction delays can be measured by evaluating simultaneous RV (Right Ventricular) and LV (Left Ventricular) IEGMs and measuring the time between the peaks of the R-waves. The goal is to characterize interatrial conduction patterns so that preload is maximized and ventricular pacing does not occur until after full closure of the mitral valve and to characterize intrinsic and paced interventricular conduction patterns so that pacing stimuli and the resultant LV and RV conduction (paced wave fronts) meet at the ventricular septum. Accordingly, QuickOpt™ Timing Cycle Optimization electrically characterizes the conduction properties of the heart to calculate optimal AV delay, PV delay (the time interval between a sensed atrial event and the ventricular impulse) and VV delay. QuickOpt™ Timing Cycle Optimization has been clinically proven to correlate with the more time-consuming echo-based methods and may be used for patients carrying CRT and dual-chamber devices at implant or follow up. QuickOpt™ Timing Cycle Optimization is an appealing optimization method since it does not require systematic measurements of a number of different AV and VV delays, which makes it very fast and simple. There are other IEGM based optimization methods among which QuickOpt™ Timing Cycle Optimization is one such method.

Despite the evident advantages of IEGM based optimization methods, such as e.g. QuickOpt™ Timing Cycle Optimization, there is an opinion within the medical community, for example, among physicians that results, e.g. timing cycles, based on input data more directly reflecting the mechanical functioning of the heart may be even more accurate and reliable. Thus, there is still a need within the art for further improved method and devices for optimizing AV and VV delays.

### SUMMARY OF THE INVENTION

An object of the present invention is to provide a system, and a method for further improvements in optimization of AV and VV delays for use in, for example, CRT.

According to an aspect of the present invention, there is provided a system including a cardiac stimulator for stimulating a heart of a patient. The cardiac stimulator is connectable to a hemodynamic sensor adapted to sense hemodynamic signals. The system further includes a pacing module arranged in the cardiac stimulator adapted to provide pacing signals for delivery to the patient via medical leads including electrodes connectable to the cardiac stimulator. A control module is arranged in the cardiac stimulator and is configured to control settings of the pacing module to deliver pacing signals in accordance with preset AV and VV delays. A data collection module is arranged in the cardiac stimulator and is configured to collect hemodynamic signals from the hemodynamic sensor during preset AV delays and VV delays. A data processing module IS arranged in the cardiac stimulator and is coupled to the data collection module. An optimization module is configured to optimize an AV and VV delay based on IEGM signals obtained via the medical leads to determine recommended AV and VV delays.

The data processing module is configured to determine a delay control parameter for each preset AV delay and VV delay based on the collected hemodynamic signals for A preset AV delay and VV delay, respectively. The data processing module is further configured to evaluate the settings for the preset AV delays and VV delays to determine the AV delay setting that corresponds to the maximum delay control parameter and the VV delay setting that corresponds to the maximum delay control parameter. The data processor is further configured to determine an AV delay error correction value as a difference between the AV delay corresponding to the maximum delay control parameter and a recommended AV delay, and to determine a VV delay error correction value as a difference between the VV delay corresponding to the maximum delay control parameter and a recommended VV delay.

According to a second aspect of the present invention, there is provided a method for a cardiac stimulator for stimulating a heart of a patient. The method includes providing and delivering pacing signals to the patient via medical leads including electrodes connectable to the cardiac stimulator, and optimizing an AV and VV delay based on IEGM signals obtained via the medical leads to determine recommended AV and VV delays The method includes delivering pacing signals in accordance with preset AV and VV delays, and sensing hemodynamic signals and collecting hemodynamic signals during preset AV delays and VV delays. The method further includes determining a delay control parameter for each preset AV delay and VV delay based on the collected hemodynamic signals for preset AV delay and VV delay, respectively. The method further evaluating the settings for the preset AV delays and VV delays to determine the AV delay setting that corresponds to the maximum delay control parameter and the VV delay setting that corresponds to the maximum delay control parameter, determining an AV delay error correction value as a difference between the AV delay corresponding to the maximum delay control parameter and a recommended AV delay, and determining a VV delay error correction value as a difference between the VV delay corresponding to the maximum delay control parameter and a recommended VV delay.

The present invention is based on the insight that an IEGM based optimization of timing cycles, such as QuickOpt™ Timing Cycle Optimization, can be further improved by using error correction values created by means of hemodynamic signals reflecting the mechanical functioning of the heart. QuickOpt™ Timing Cycle Optimization is based on the hypothesis that the point of time for mitral valve closure can be estimated by measuring the interatrial conduction time (P-wave duration), that onset of isovolumetric contraction can be measured using the peak of the R-wave, and that interventricular conduction delays can be measured by evaluating simultaneous RV and LV IEGM:s and measuring the time between peaks of the R-waves.

Since the AV and VV delays calculated using IEGM based optimization such as QuickOpt™ Timing Cycle Optimization are partly based on a hypothetical foundation, the inventors have realized that the AV and VV delays can be made more accurate by using error correction parameters based on hemodynamic signals, which directly reflects the mechanical functioning of the heart.

In fact, the optimization can be improved in a number of different ways by including hemodynamic data and modifying the AV and VV delays based on parameters calculated using this hemodynamic data.

For example, studies have shown that AV and VV delays based on hemodynamic signals may be more reliable and accurate than IEGM based delays. For example, there are studies showing a poor correlation between IEGM based delays and delays based on pressure measurements, e.g. LV dP/dtmax, (“The Optimized V-V interval Determined by Interventricular Conduction Times versus Invasive Measurement by LV dP/dtmax”, van Gelder, et al.). Hence, the reliability and accuracy of the AV and VV delays, and thus the CRT, can be improved using the combined IEGM and hemodynamically based optimization as suggested by the present invention. Furthermore, the IEGM based optimizations, such as e.g. QuickOpt™ Timing Cycle Optimization, may not deliver the optimal AV and VV delays for all patients due to the fact that they are to some extent based on a hypothesis created from average values and parameters collected from a large number of patients. Thereby, there might be individual patients for whom the IEGM based AV and VV delays are not suitable. By adapting and tailoring the IEGM based delays with data from the hemodynamic measurements obtained from the specific patient after implantation, better AV and VV delays adapted for that patient can be created.

Further, the invention has also been developed taking into consideration the problems associated with hemodynamic sensors in a chronic setting. An optimization of AV and VV delays based only on hemodynamic data may not be reliable due to problems related to, for example, overgrowth and clogging, which may lead to a drift of the sensor signals delivered.

Moreover, hemodynamic sensors are often also sensitive to the physical placement. An erroneous or less optimal placement of a sensor (or lead) may lead to a perturbed signal.

Further, hemodynamic sensors are often sensitive to motion artifacts and body-noise.

In addition, an optimization based only on hemodynamic data would probably lead to high power consumption since the hemodynamic sensor generally has relatively high power consumption.

In view of this, the present invention is also based on the idea of determining AV and VV delay, for example, for CRT by combining the IEGM based optimization with results based on hemodynamic data, and by modifying the AV and VV delays from the IEGM based optimization with correction values obtained based on hemodynamic data.

Thus, the present invention makes use of the advantages of the IEGM based optimization and hemodynamically based sensor data, respectively. This is achieved in that the IEGM based optimization is used to determine rough initial or start values for the AV and VV delay, which can be done in a prompt and quick way. The more time consuming and power consuming hemodynamic data collection is then performed to create correction values to the initially determined AV and VV delays. The correction values can also be iteratively improved over time.

According to embodiments of the present invention, the recommended AV and VV delay are updated with the AV and VV delay error correction value, respectively, to obtain a modified recommended AV and VV delay. These modified AV and VV delays may be further redefined in an iterative process, i.e. by iteratively modifying AV and VV delay error correction values with new AV and VV delay error correction values obtained in successive procedures. That is, current AV and VV delay error correction values are modified using at least one set of AV and VV delay error correction values obtained from a subsequent determination of AV and VV delay error correction values.

According to an embodiment of the present invention, the pacing module is controlled to deliver pacing signals in accordance with preset VV delays at a specific AV delay, the specific AV delay being the AV delay corresponding to the maximum delay control parameter. Moreover, a delay control parameter for each VV delay is determined based on the collected hemodynamic signals for respective VV delay obtained at the specific AV delay; and the settings for the VV delays are evaluated to determine the VV delay setting that corresponds to the maximum delay control parameter at the specific AV delay. This provides a quicker and more efficient algorithm in terms of calculation time and battery consumption since a fewer number of VV delay measurements have to be performed and hence a fewer number of delay control parameters have to be determined.

According to embodiments of the present invention, the hemodynamic sensor is an implantable pressure sensor. In specific embodiments, the pressure sensor is adapted for transmural placement so as to enable sensing of a left ventricular pressure. A suitable pressure sensor is, for example described, in the co-pending application PCT/EP2010/058624 by the same applicant, and in US RE 39,863, U.S. Pat. No. 6,248,083, or US RE 35,648, all herein incorporated by reference. If the pressure sensor as described in the co-pending application PCT/EP2010/058624 is used, it is possible to implant the pressure sensor without any punching/drilling of hole in the septum and it is possible to implant the pressure sensor transseptally from the right ventricle to the left ventricle. The pressure sensor can be placed fast and without any additional tools required than what is normally used for lead implantation. The sensor can easily be pushed through the myocardium for access to the left side with minimal damage to the tissue of the septum. This pressure sensor however has a limited (less than a year) useful life due to, for example, overgrowth.

According to embodiments of the present invention, the hemodynamic sensor is selected from the group including: accelerometers, blood flow probes, load indicators which react to geometrical changes, heart sound sensors, and photoplethysmography sensors.

In embodiments of the present invention, the cardiac stimulator further comprises a communication module adapted to communicate with extracorporeal equipment wirelessly using RF or inductive telemetry.

According to embodiments of the present invention, the optimization module is arranged in extracorporeal equipment having a communication module for communication with the communication module of the cardiac stimulator.

In other embodiments of the present invention, the optimization module is arranged in the cardiac stimulator.

According to embodiments of the present invention, the left ventricle pressure derivative is calculated for each AV and VV delay, wherein the delay control parameter is the left ventricle pressure derivative.

According to an embodiment of the present invention, a specific heart rate interval based on the sensed heart rate at which the hemodynamic signals were obtained is determined. Thereby, according to some embodiments, an approximate heart rate or heart rate interval is associated with or labeled to each AV or VV error correction parameter. The AV and VV delay error correction values may be binned together according to what approximate heart rate or heart rate interval at which they were acquired. For example, if a first procedure to obtain AV and VV delay error correction values were performed between 65-75 bpm, and a second procedure to obtain AV and VV delay error correction values were performed between 75-85 bpm, the results from these procedures will not be mixed. Instead, this will result in two sets of error correction values, one set of AV and VV delay error correction values of the heart rate interval between 65-75 bpm and one set of AV and VV delay error correction values of the heart rate interval between 75-85 bpm. Thus, it is possible to obtain heart rate specific error correction values and, in turn, heart rate specific AV and VV delays. Thereby, it is possible to obtain rate dependent error correction terms and AV and VV delays.

### BRIEF DESCRIPTION OF THE DRAWINGS

FIG. 1 is a simplified diagram of one embodiment of an implantable stimulation device in electrical communication with several leads implanted in a patient's heart for measuring hemodynamic signals delivering multi-chamber stimulation.

FIG. 2 is a simplified functional block diagram of one embodiment of a system in accordance with the present invention, illustrating basic elements of the system.

FIG. 3 is a simplified functional block diagram of another embodiment of a system in accordance with the present invention, illustrating basic elements of the system.

FIG. 4 is a flow chart illustrating general steps of a method for optimizing AV and VV delays according to the present invention.

FIG. 5 is a flow chart illustrating steps of an embodiment of the method for optimizing AV and VV delays according to the present invention.

### DESCRIPTION OF THE PREFERRED EMBODIMENTS

The following is a description of exemplifying embodiments in accordance with the present invention. This description is not to be taken in limiting sense, but is made merely for the purposes of describing the general principles of the invention. It is to be understood that other embodiments may be utilized and structural and logical changes may be made without departing from the scope of the present invention. Some embodiments of the present invention relate generally to implantable pressure sensors but, however, even though particular types of pressure sensors are mentioned herein, the present invention is not limited to pressure sensors but may include other types of hemodynamic sensors such as, accelerometers, blood flow probe, load indicators which react to geometrical changes, heart sound sensors, or photoplethysmography sensors or similar sensors.

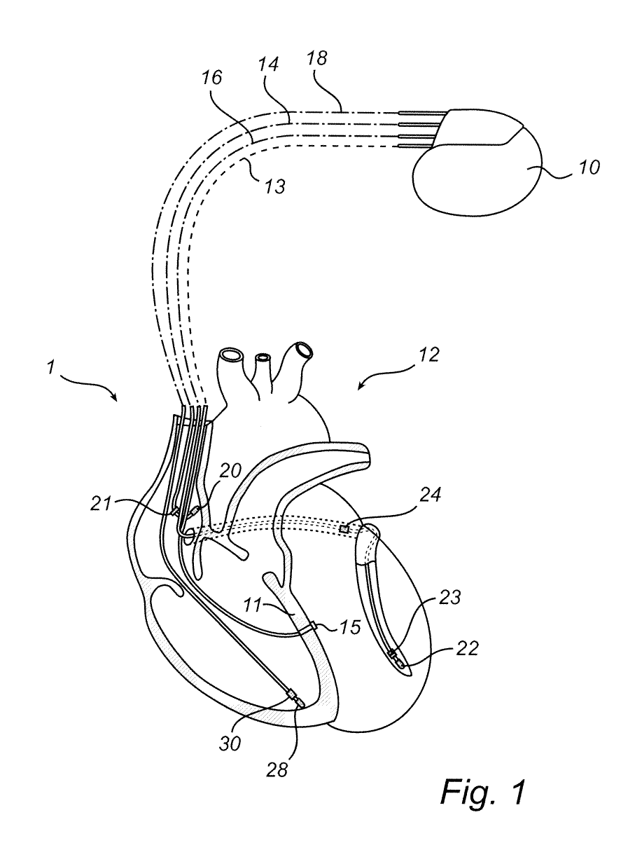

Referring to FIG. 1, one implementation of the present invention relating to a system including an implantable cardiac stimulator connectable to one or more medical leads will be discussed. In this embodiment the cardiac stimulator is described as being connectable to an implantable pressure sensor but, as mentioned above, there are a number of other hemodynamic sensors that may be conceivable.

The implantable cardiac stimulator 10 of the system 1 is in electrical communication with a patient's heart 12 by way of three leads 14, 16, and 18 suitable for delivering multi-chamber stimulation therapy. Further, the cardiac stimulator 10 is connected to a hemodynamic sensor 15, which in one particular embodiment of the present invention is a pressure sensor 15 attached to septum 11 arranged in a medical lead 13 coupled to the cardiac stimulator 10. A suitable pressure sensor is, for example described, in the co-pending application PCT/EP2010/058624 by the same applicant, US RE 39,863, U.S. Pat. No. 6,248,083, and US RE 35,648, incorporated herein by reference. However, as mentioned above, other types of hemodynamic sensors may alternatively or as a complement be used including accelerometers for measuring pressure changes in the left ventricle, flow probes, load indicators for measuring geometrical changes in the cardiac tissue e.g. in septum, heart sound sensors, or photoplethysmographic sensors.

To sense atrial signals and to provide right atrial chamber stimulation therapy, the stimulator 10 is coupled to an implantable right atrial lead 14 having, for example, an atrial tip electrode 20, which typically is implanted in the patient's right atrial appendage or septum. FIG. 1 shows the right atrial lead 14 as also having an atrial ring electrode 21.

To sense left atrial and ventricular cardiac signals and to provide left chamber pacing therapy the stimulator is coupled to a coronary sinus lead 16 designed for placement in the coronary sinus region via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible via the coronary sinus.

The lead 16 is designed to receive atrial and ventricular cardiac signals and to deliver left ventricular pacing therapy using, for example, a left ventricular tip electrode 22, a left ventricular ring electrode 23, and left atrial pacing therapy using, for example, a left atrial ring electrode 24.

The cardiac stimulator 10 is also in electrical communication with the heart 12 by way of an implantable right ventricular lead 18 having, in this embodiment, a right ventricular tip electrode 28 and a right ventricular ring electrode 30. Typically, the right ventricular lead 18 is transvenously inserted into the heart 12 to place the right ventricular tip electrode 28 in the right ventricular apex. The right ventricular lead 18 is capable of sensing or receiving cardiac signals, and delivering stimulation in the form of pacing therapy.

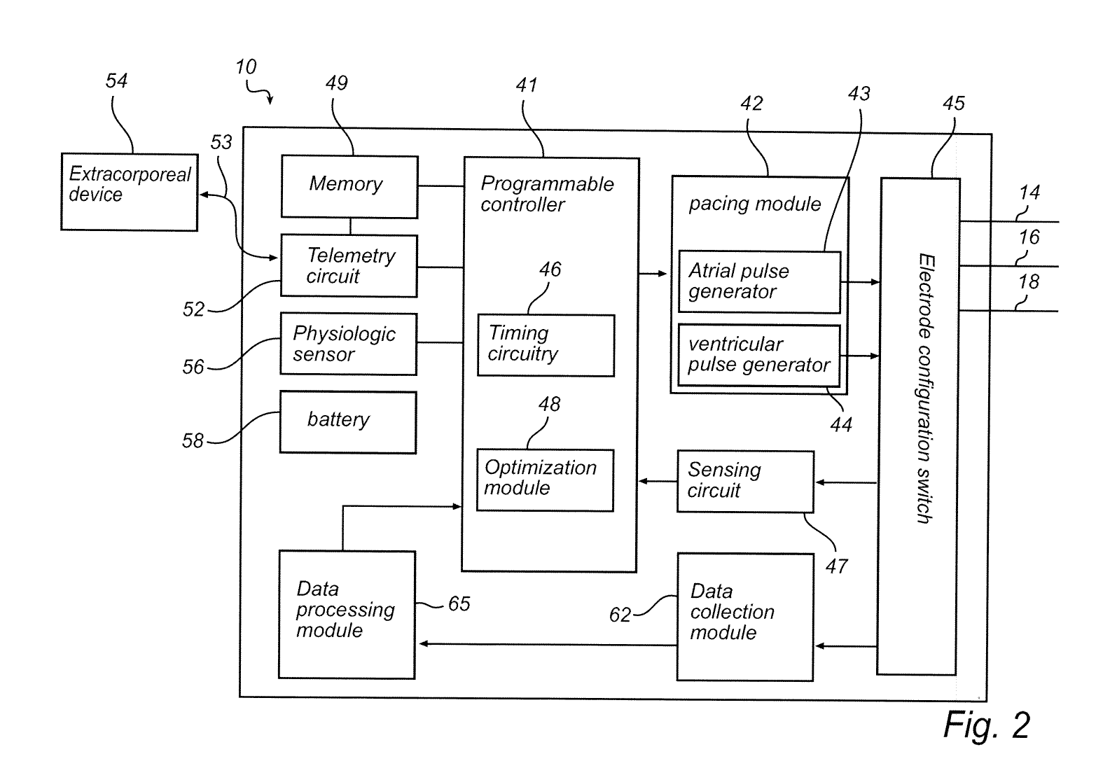

In FIG. 2, an exemplary, simplified block diagram depicting various components of the cardiac stimulator according to embodiments of the present invention is shown. The cardiac stimulator 10 is capable of delivering cardiac resynchronization therapy and is configured to integrate both monitoring and therapy features, as will be described below. The cardiac stimulator 10 collects and processes data about the heart 12 from one or more sensors including at least one hemodynamic sensor 15. In one particular embodiment of the present invention, the hemodynamic sensor is a pressure sensor 15 arranged in a medical lead 13 connectable to the cardiac stimulator 10, see FIG. 1. Further, the cardiac stimulator 10 collects and processes data about the heart 12 from electrode pairs for sensing cardiac electrogram (EGM) signals. While a particular multi-chamber device is shown, it is to be appreciated and understood that this is done for illustration purposes only. Thus, the techniques and methods described below can be implemented in connection with any suitable configured or configurable stimulation device. Accordingly, one of skill in the art could readily duplicate, eliminate, or disable the appropriate circuitry in any desired combination to provide a device capable of treating the appropriate chamber with pacing stimulation including cardiac resynchronisation therapy.

The cardiac stimulator 10 has a housing 40, often referred to as the “can” or “case electrode”. The housing 40 may function as a return electrode in “unipolar” modes. Further, the housing 40 includes connector (not shown) having a plurality of terminals (not shown) for connection with electrodes and/or sensors.

The cardiac stimulator 10 includes a programmable microcontroller or control module 41 that inter alia controls the various modes of stimulation therapy. As well known within the art, the microcontroller 41 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 41 includes the ability to process or monitor input signals (data or information) as controlled by a program stored in a designated block of memory. The type of microcontroller is not critical to the described implementations. Rather, any suitable microcontroller 41 may be used that carries out the functions described herein. The use of micro-processor based control circuits for performing timing and data analysis are well known in the art.

Furthermore, the cardiac stimulator 10 includes pacing module 42 adapted to provide pacing signals for delivery to the patient. The pacing module 42 comprises an atrial pulse generator 43 and a ventricular pulse generator 44 that generate pacing stimulation pulses for delivery by the right atrial lead 14, the coronary sinus lead 16, and/or the right ventricular lead 18 via an electrode configuration switch 45. It is understood that in order to provide stimulation therapy in each of the four chambers, the atrial and ventricular pulse generators 43 and 44, may include dedicated, independent pulse generators, multiplexed pulse generators, or shared pulse generators. The pulse generators 43 and 44 are controlled by the microcontroller 41 via appropriate control signals to trigger or inhibit stimulation pulses.

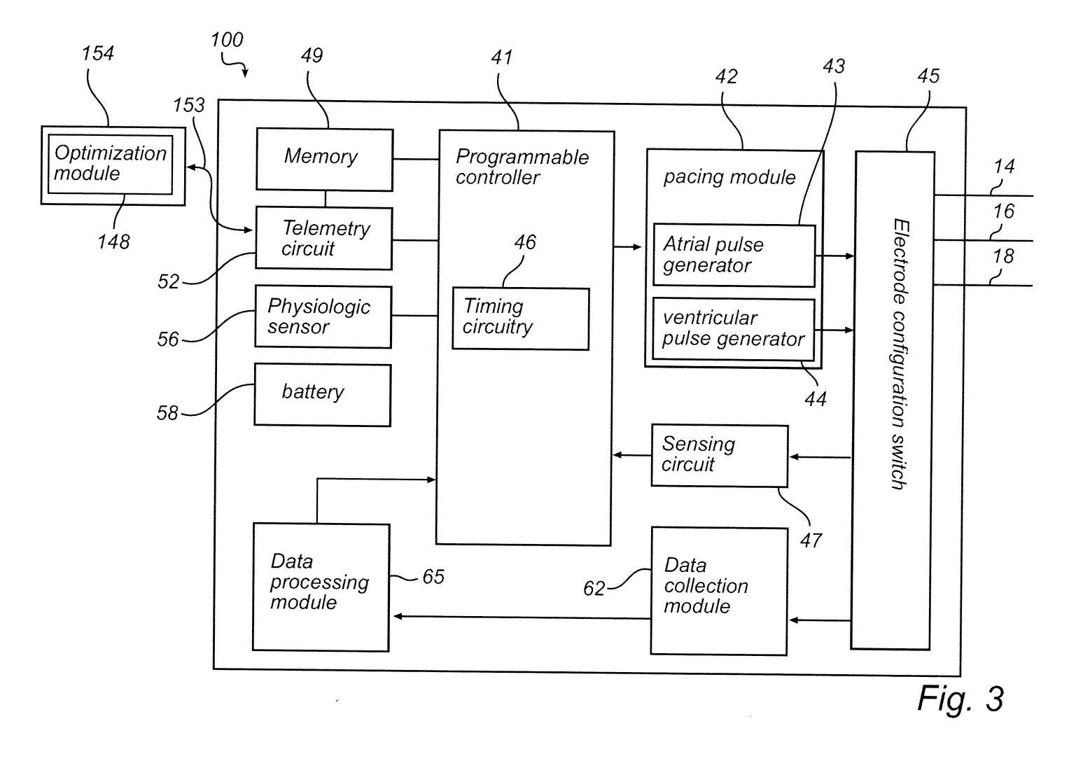

The microcontroller 41 further includes timing control circuitry 46 to control timing of the stimulation pulses (e.g. pacing rate, AV delay, VV delay, etc.) as well as to keep track of timing of refractory periods blanking intervals, etc. which is well known in the art. In addition, the microcontroller 41 may include components such as e.g. an arrhythmia detector (not shown) and/or a morphology detector (not shown). Moreover, an optimization module 48 is adapted to optimize an AV and VV delay based on IEGM signals to determine recommended AV and VV delays. In embodiments of the present invention, the optimization module 48 employs QuickOpt™ Timing Cycle Optimization for IEGM based AV timing optimization and VV timing optimization. The aforementioned components may be implemented as part of the microcontroller 41, or as software/firmware instructions programmed into the device and executed on the microcontroller 41 during certain modes of operation. In alternative embodiments the optimization module is arranged in an extracorporeal or external device instead of being arranged in the cardiac stimulator. One example of such an embodiment is shown in FIG. 3. In the embodiment shown in FIG. 3, the cardiac stimulator is denoted by the reference FIG. 100. The cardiac stimulator 100 has an optimization module 148 arranged in an extracorporeal device 154.

A sensing circuit 47 comprising atrial sensing circuits and ventricular sensing circuits may also be coupled to the right atrial lead 14, the coronary sinus lead 16, and the right ventricular lead 18 through the switch 45 for detecting the presence of cardiac activity in each of the four chambers of the heart. Accordingly, the atrial sensing circuits and ventricular sensing circuits 47 may include dedicated sense amplifiers, multiplexed amplifiers, or shared amplifiers.

The output from the atrial sensing circuits and ventricular sensing circuits 47 are connected to the microcontroller 41, which, in turn, is able to trigger or inhibit the atrial sensing circuits and ventricular sensing circuits 47 in a demand fashion in response to the absence or presence of cardiac activity in the appropriate chamber of the heart.

Furthermore, the microcontroller 41 is coupled to a memory 49 by a suitable data/address bus (not shown), wherein the programmable operating parameters used by the microcontroller 41 are stored and modified, as required, in order to customize the operation of the cardiac stimulator to the needs of a particular patient. Such operating parameters define, for example, pacing pulse amplitude, pulse duration, etc. Advantageously, the operating parameters may be non-invasively programmed into the memory 49 through a communication module 52 including, for example, a telemetry circuit for telemetric communication via communication link 53 with an external device 54, such as a programmer or a diagnostic system analyzer. The telemetry circuit advantageously allows intracardiac electrograms and status information relating to the operation of the device 10 to be sent to the external device 54 through an established communication link 53. Further, the communication module may alternatively or as a complement to the telemetry circuit include circuit for RF communication.

The cardiac stimulator 10 may further include a physiologic sensor 56, commonly referred to as a “rate-responsive” sensor because it is typically used to adjust pacing stimulation rate according to the exercise state of the patient. While shown as being included within the stimulator 10, it is to be understood that the physiologic sensor 56 also may be external to the stimulator, yet still be implanted within or carried by the patient. Examples of physiologic sensors include sensors that, for example, sense respiration rate, or activity variance.

Moreover, the cardiac stimulator 10 additionally includes a battery 58 that provides operating power to all of the circuits shown in FIG. 2. Preferably, the stimulator 10 employs lithium or similar battery technology.

A data collection module 62 is adapted to collect hemodynamic signals from the hemodynamic sensor 15, for example, during different AV delays and VV delays for later processing of the data. The data collection module 62 comprises, for example, analog-to-digital (A/D) data acquisition circuits adapted to acquire and amplify the signals from the hemodynamic sensor 15 and to convert the raw analog data into a digital signal, filter the signals and store the digital signals for later processing in a data processing module 65, which also may be integrated in the microcontroller 41.

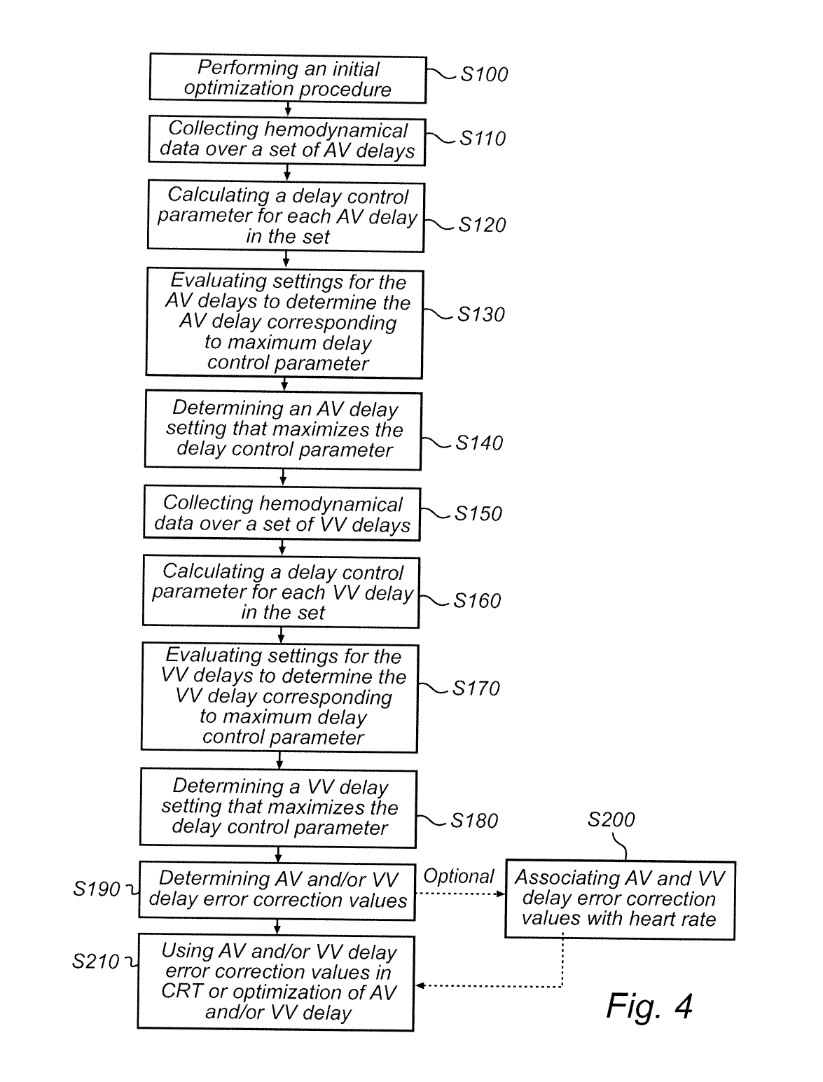

According to embodiments of the present invention, the data processing module 65 is adapted to determine or calculate delay control parameters for AV delays and VV delays based on the collected hemodynamic signals for respective AV delay and VV delay as will be described in more detail below with reference to FIG. 4. These delay control parameters are used to evaluate the settings for the AV delays and VV delays to determine the AV delay setting that corresponds to the maximum delay control parameter and the VV delay setting that corresponds to the maximum delay control parameter. Based on this, an AV delay error correction value is determined as a difference between the AV delay corresponding to the maximum delay control parameter and a recommended AV delay and a VV delay error correction value is determined as a difference between the VV delay corresponding to the maximum delay control parameter and a recommended VV delay. The recommended AV and VV delay are, for example, determined by means of the optimization module 48 employing QuickOpt™ Timing Cycle Optimization.

With reference now to FIG. 4, one embodiment of the optimization procedure for obtaining timing data for CRT according to the present invention will be described. First, at step S100, an optimization based on IEGM data is performed to obtain AV and VV timings, for example, the QuickOpt™ Timing Cycle Optimization, which is an algorithm that provides IEGM based AV timing optimization in CRT and ICD systems and VV timing optimization in CRT devices. QuickOpt™ Timing Cycle Optimization provides recommended AV and VV delays: AVD_{QO} and VVD_{QO}, respectively. Optionally, the heart rate is also stored with a timestamp linking it to the recommended AV and VV delays. The optimization includes providing and delivering pacing signals to the patient via the medical leads 14, 16, 18.

Thereafter, at step S110, a sweep across a preset or predetermined set of AV delays is initiated and the hemodynamic signal (or signals) is simultaneously recorded during each AV delay. In one preferred embodiment, the hemodynamic signal is the left ventricular pressure (LVP). One example of such AV delay (AVD) sweep could be the following: AVD₁=60 ms, AVD₂=80 ms, AVD₃=100 ms, ADV₄=120 ms, AVD₅=140 ms, AVD₆=160 ms, AVD₇=180 ms.

For each of these 20 ms delays a sequence of hemodynamic data will be recorded. That is the following hemodynamic data (hd) is recorded: hd₁, hd₂, hd₃, hd₄, hd₅, hd₆, hd₇. In one particular embodiment, the left ventricular pressure is recorded and accordingly: LVP₁, LVP₂, LVP₃, LVP₄, LVP₅, LVP₆, LVP₇.

Subsequently, at step S120, a delay control parameter is calculated for each AV delay. According to one particular embodiment, the left ventricular pressure (LVP) signal is bandpass filtered and differentiated to produce a continuous dLVP/dt signal: dLVP₁/dt, dLVP₂/dt, dLVP₃/dt, dLVP₄/dt, dLVP₅/dt, dLVP₆/dt, dLVP₇/dt.

At step S130, the settings for the AV delays are evaluated to determine the AV delay setting that corresponds to the maximum delay control parameter. In one embodiment of the present invention, each sequence of dLVP/dt data will be processed to identify each local maxima of the differentiated LVP signal (i.e. dLVP/dt_{MAX}). In this specific embodiment, the following values are created: dLVP₁/dt_{MAX}, dLVP₂/dt_{MAX}, dLVP₃/dt_{MAX}, dLVP₄/dt_{MAX}, dLVP₅/dt_{MAX}, dLVP₆/dt_{MAX}, dLVP₇/dt_{MAX}.

Thereafter, at step S140, the setting that maximizes the delay control parameter is determined. In the discussed embodiment, the setting that maximizes the differentiated LVP (i.e. max (dLVP/dt_{MAX})) is determined. According to an embodiment of the present invention, the setting that maximizes the delay control parameter is identified by fitting a 2^{nd} or 3^{rd} degree polynomial to the data points using least squares. The setting that maximizes the delay control parameter is denoted AVₚ.

At step S150, a second sweep is performed over different (which may be preset or predetermined) VV delays is performed, for example, at AVD=AVₚ. To counteract drift, a control measurement where VVD is set to zero is performed between each VVD measurement. According to an example, the sweep can be as follows:

VV₁=−80 ms, VV=0; VV₂=−60 ms, VV=0; VV₃=−40 ms, VV=0; VV₄=−20 ms, VV=0; VV₅=0; VV₆=20 ms, VV=0; VV₇=40 ms, VV=0; VV₈=60 ms, VV=0; VV₁=80 ms, VV=0.

Hemodynamic signal (or signals) is simultaneously recorded during each VV delay. In one preferred embodiment, the hemodynamic signal is the left ventricular pressure (LVP).

At step S160, a delay control parameter is determined for each VV delay. According to one particular embodiment, the left ventricular pressure (LVP) signal is bandpass filtered and differentiated to produce a continuous dLVP/dt signal: dLVP₁/dt, dLVP₂/dt, dLVP₃/dt, dLVP₄/dt, dLVP₅/dt, dLVP₆/dt, dLVP₇/dt, dLVP₈/dt, dLVP₉/dt.

Thereafter, at step S170, the VV delay settings are evaluated to determine the VV delay setting that corresponds to the maximum delay control parameter. In one embodiment of the present invention, each sequence of dLVP/dt data will be processed to identify each local maxima of the differentiated LVP signal (i.e. dLVP/dt_{MAX}). In this specific embodiment, the following values are created: dLVP₁/dt_{MAX}, dLVP₂/dt_{MAX}, dLVP₃/dt_{MAX}, dLVP₄/dt_{MAX}, dLVP₅/dt_{MAX}, dLVP₆/dt_{MAX}, dLVP₇/dt_{MAX}, dLVP₈/dt_{MAX}, dLVP₉/dt_{MAX}.

At step S180, the setting that maximizes the delay control parameter is determined. In the discussed embodiment, the setting that maximizes the differentiated LVP (i.e. max (dLVP/dt_{MAX})) is determined. According to an embodiment of the present invention, the setting that maximizes the delay control parameter is identified by fitting a 2^{nd} or 3^{rd} degree polynomial to the data points using least squares. The setting that maximizes the delay control parameter is denoted VVₚ.

Subsequently, at step S190, AV delay error correction values and VV delay error correction values are determined as a difference between the AV and VV delay corresponding to the maximum delay control parameter and a recommended AV delay and VV delay respectively. That is, AV delay error correction values and VV delay error correction values are determined according to:

delta_{AV}=AVD_{QO}−AV_{P}

delta_{VV}=VVD_{QO}−VV_{P}

Then, at step S210, the AV and VV delay error correction values are used in CRT or further optimization for update of the AV and VV delays. Optionally, at step 200, the delta values (delta_{AV} and delta_{VV}) are labeled or associated with an approximate heart rate or heart rate interval during which they were obtained. Accordingly, step S200 is optional and if not used, the algorithm proceeds directly to step S210 from step S190.

If the error correction values are associated with or labeled to each AV or VV error correction parameter, it is possible to obtain rate dependent error correction terms and AV and VV delays. The AV and VV delay error correction values may be binned together according to what approximate heart rate or heart rate interval at which they were acquired. For example, if a first procedure to obtain AV and VV delay error correction values were performed between 65-75 bpm, and a second procedure to obtain AV and VV delay error correction values were performed between 75-85 bpm, the results from these procedures will not be mixed. Instead, this will result in two sets of error correction values, one set of AV and VV delay error correction values of the heart rate interval between 65-75 bpm and one set of AV and VV delay error correction values of the heart rate interval between 75-85 bpm. Thus, it is possible to obtain heart rate specific error correction values and, in turn, heart rate specific AV and VV delays.

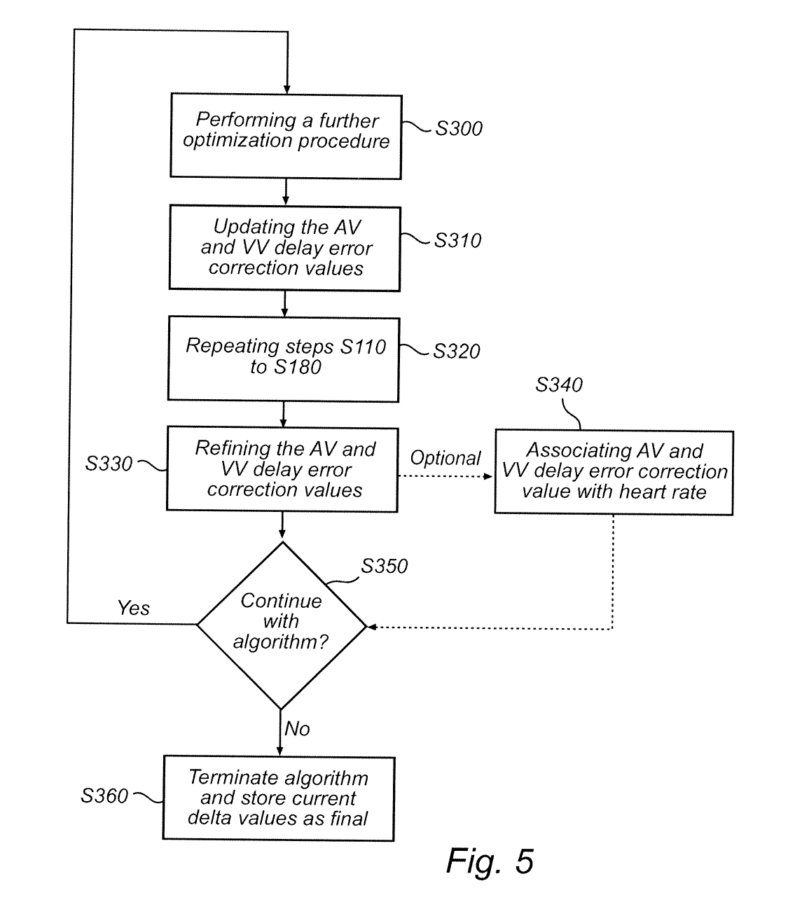

With reference now to FIG. 5, steps performed in block S210 according to an embodiment of the present invention will be described.

First, at step S300, a further optimization procedure is performed at a later point of time, for example, a week later than the first optimization procedure. Thus, an optimization based on IEGM data is performed to obtain AV and VV timings, for example, the QuickOpt™ Timing Cycle Optimization, QuickOpt™ Timing Cycle Optimization provides recommended AV and VV delays: AVD_{QO2} and VVD_{QO2}, respectively. Optionally, the heart rate is also stored with a timestamp linking it to the recommended AV and VV delays. However, the values obtained by means of the optimization procedure is not stored as recommended values but, at step S310, these values are updated with the AV and VV delay error correction values, respectively, according to the following:

AVD_{QO-updated}=AVD_{QO2}−delta_{AV}

VVD_{QO-updated}=AVD_{QO2}−delta_{AV}

Thereafter, at step S320, the steps S110-S180 are repeated with the updated AV and VV delays as input values.

At step S330, the AV and VV delay error correction values are redefined using the delta_{AV} and delta_{VV} from the latest algorithm loop according to the following:

Delta_{AV}_{_{—}}_{redefined}=delta_{AV}_{_{—}}_{old}+delta_{AV}_{_{—}}_{new}

Delta_{VV}_{_{—}}_{redefined}=delta_{VV}_{_{—old}}+delta_{VV}_{_{—}}_{new}

Accordingly, the delta values from the latest loop are added to the respective delta values from the previous loop. The redefined delta values may optionally be associated or labeled with an approximate heart rate or heart rate interval during which they were obtained at step S340. Accordingly, step S340 is optional and if not used, the algorithm proceeds directly to step S350 from step S330. At step S350, it is checked whether a further optimization should be performed to obtain a new set of updated delta values or if the algorithm should be terminated. If it is determined that a further optimization should be performed, the algorithm returns to step S300. If it is determined that the algorithm should be terminated, the current delta values may be stored as final values for use in the CRT at step S360. For example, if it is verified that the hemodynamic sensor 15 not provides reliable data any longer, e.g. due to over growth, a decision to terminate the algorithm can be made.

Although certain embodiments and examples have been described herein, it will be understood by those skilled in the art that many aspects of the devices and methods shown and described in the present disclosure may be differently combined and/or modified to form still further embodiments. Alternative embodiments and/or uses of the devices and methods described above and obvious modifications and equivalents thereof are intended to be within the scope of the present disclosure. Thus, it is intended that the scope of the present invention should not be limited by the particular embodiments described above.

I claim as my invention:

1. A system comprising:
an implantable cardiac stimulator for stimulating a heart of a patient, said cardiac stimulator;
a hemodynamic sensor connected to said cardiac stimulator, that senses hemodynamic signals;
medical leads including electrodes connected to the cardiac stimulator;
a pacing module in said cardiac stimulator configured to provide pacing signals for delivery to said patient via said leads electrodes;
a control module in said cardiac stimulator configured to control settings of said pacing module to deliver said pacing signals in accordance with preset AV and VV delays;
a data collection module in said cardiac stimulator configured to collect hemodynamic signals from said hemodynamic sensor during said preset AV delays and VV delays;
a data processing module in said cardiac stimulator in communication with said data collection module;
an optimization module configured to optimize an AV and VV delay based on IEGM signals obtained via said medical leads to determine recommended AV and VV delays; and
said data processing module being configured to determine a delay control parameter for each preset AV delay and VV delay based on the collected hemodynamic signals for respective preset AV delay and VV delay, evaluate the settings for said preset AV delays and Vv delays to determine the AV delay setting that corresponds to the maximum delay control parameter and the VV delay setting that corresponds to the maximum delay control parameter, determine an AV delay error correction value as a difference between the AV delay corresponding to the maximum delay control parameter and a recommended AV delay, and determine a VV delay error correction value as a difference between the VV delay corresponding to the maximum delay control parameter and a recommended VV delay.

2. The system according to claim 1 wherein said data processing module is configured to update said recommended AV and/or VV delay with said AV and/or VV delay error correction value, respectively, to obtain a modified recommended AV and/or VV delay.

3. The system according to claim 1 wherein said data processing module is configured to modify current AV and VV delay error correction values using at least one set of AV and VV delay error correction values obtained from a subsequent determination of AV and VV delay error correction values.

4. The system according to claim 1 wherein:
said control module is configured to control said pacing module to deliver pacing signals in accordance with preset VV delays at a specific AV delay, said specific AV delay being the AV delay corresponding to the maximum delay control parameter; and
said data processing module is configured to determine a delay control parameter for each VV delay based on the collected hemodynamic signals for respective VV delay obtained at said specific AV delay, and evaluate the settings for the VV delays to determine the VV delay setting that corresponds to the maximum delay control parameter at said specific AV delay.

5. The system according to claim 1 wherein said hemodynamic sensor is an implantable pressure sensor.

6. The system according to claim 5 wherein said pressure sensor is configured for transmural placement so as to enable sensing of a left ventricular pressure.

7. The system according to claim 1 wherein said hemodynamic sensor is selected from the group including: accelerometers, blood flow probes, load indicators which react to geometrical changes, heart sound sensors, and photoplethysmography sensors.

8. The system according to claim 1 wherein said cardiac stimulator further comprises a communication module configured to communicate with extracorporeal equipment wirelessly using RF or inductive telemetry.

9. The system according to claim 8 wherein said optimization module is located in extracorporeal equipment having a communication module for communication with said communication module of said cardiac stimulator.

10. The system according to claim 1 wherein said optimization module is located in said cardiac stimulator.

11. The system according to claim 1 wherein said pressure sensor is configured for transmural placement to enable sensing of left ventricular pressure, and wherein said data processing module is configured to calculate a left ventricle pressure derivative for each AV and VV delay, and wherein said delay control parameter is said left ventricle pressure derivative.

12. The system according to claim 1 wherein said data processing module is configured to determine a heart rate or heart rate interval based on the sensed heart rate at which the hemodynamic signals were obtained.

13. The system according to claim 12, wherein said data processing module is configured to associate a heart rate or heart rate interval with each AV and/or VV error correction parameter.
